(19) United States
(12) Patent Application Publication
(10) Pub. No.: US 2018/0015308 A1
(43) Pub. Date: Jan. 18, 2018

(54) ULTRASOUND BASED COSMETIC THERAPY METHOD AND APPARATUS

(71) Applicant: JeNu Biosciences, LLC, Hermosa Beach, CA (US)

(72) Inventors: Justin REED, Seattle, WA (US); Alexander LEBEDEV, Seattle, WA (US); Michael LAU, Edmonds, WA (US); George BARRETT, Redmond, WA (US); Irena LEBEDEV, Upland, CA (US)

(21) Appl. No.: 15/674,714

(22) Filed: Aug. 11, 2017

Related U.S. Application Data
(63) Continuation of application No. 15/357,295, filed on Nov. 21, 2016, now abandoned.
Application No. 15/357,295 is a continuation of application No. 13/400,027, filed on Feb. 17, 2012, now abandoned.
Application No. 13/400,027 is a continuation of application No. 12/487,538, filed on Jun. 18, 2009, now abandoned.
(60) Provisional application No. 61/073,670, filed on Jun. 18, 2008.

(51) Int. Cl. A61N 7/00; A61M 35/00; A61M 37/00

(52) U.S. Cl. CPC A61N 7/00; A61M 37/0092; A61M 35/003; A61N 2007/0039; A61N 2007/0034

(57) ABSTRACT

A waveguide couples an acoustic source (such as an ultrasound transducer) to a custom cosmetic product (e.g., a liquid or gel-based skin care product) applied to the skin. In one exemplary embodiment, a distal surface of the waveguide is placed in contact with a relatively thin layer of skin care product that has been applied to the skin. Alternatively, the thin layer can be applied to the distal face of the waveguide, and then the waveguide placed on the skin. The custom cosmetic product has been formulated such that when ultrasound energy is directed into the custom cosmetic product via the waveguide, bubbles included or formed in the custom cosmetic product oscillate, and increase the permeability of the skin to active beneficial agents included in the custom cosmetic product.

### RELATED APPLICATIONS

This application is based on a prior copending provisional application, Ser. No. 61/073,670, filed on Jun. 18, 2008, the benefit of the filing date of which is hereby claimed under 35 U.S.C. §119(e).

### BACKGROUND

Human skin inevitably deteriorates with age. The skin of a young child is typically smooth, firm, unwrinkled, evenly colored, and blemish free. As one ages, the skin becomes rough, dry, lax, wrinkled, and irregular in color and pigmentation. The skin deterioration is due to intrinsic aging and photoaging. Also, abnormalities in the pilosebaceous units and dysfunction of the melanocyste/keratinocyte units contribute to the skin deterioration. Extrinsic factors, such as sunlight, tanning UV light, makeup and improper use of moisturizers can further aggravate the intrinsic aging process of the skin. There are various treatment modalities to attempt to stop or reverse the skin aging process.

The various treatment modalities fall into two basic categories. First, using chemical products, one attempts to condition the skin by increasing its tolerance to damage, to correct the defects of the epidermal layer, and to stimulate the basal layer of the epidermis and papillary dermis to improve skin function. Second, using physical or chemical means, one attempts to remove the deteriorated epidermal and dermal tissue to allow the replacement with new skin of more normal and desirable characteristics. These chemical and physical agents include, for example: chemical peels such as TCA, dermabrasions, lasers, ionic plasma, etc. The effectiveness and side effects of the various modalities might or might not correlate with the invasiveness of the processes. In general terms, though, it is reasonable to suggest that most people would prefer processes that are not invasive, that are safe, and that are reasonably effective to treat their skin. The chemical products designed to condition, correct, or stimulate the skin in lotion or gel form are non-invasive. These products, if formulated properly, are relatively safe. However, the effectiveness of these products is often questionable. The epidermis, especially the horny layer of the stratum corneum, functions as a barrier to prevent penetration by any external fluids into the body. Unless the therapeutic chemicals can get to the basal layer of the epidermis and the papillary dermis, they cannot affect the keratinocyte or melanocyte function to improve the epidermal appearance and texture. It is even more difficult for topically-applied therapeutic chemicals to affect the deeper dermal tissue where the collagen, elastic fibers, and extracellular matrix largely determine the look and feel of the skin.

It would thus be desirable to provide a more effective method and apparatus to improve the look and feel of the skin. Further, it would be preferable to employ a non-invasive procedure to achieve these results.

### SUMMARY

This application specifically incorporates by reference the disclosure and drawings of the provisional patent application identified above as a related application.

The concepts disclosed herein address the above-mentioned problems by using ultrasound to enhance the penetration of a therapeutic agent into the epidermis and dermis, in a non-invasive process, to achieve conditioning, correction and stimulation of the skin, to improve its appearance and feel.

In a basic exemplary embodiment, a waveguide couples an acoustic source (such as an ultrasound transducer) to a custom cosmetic product (i.e., a liquid- or gel-based skin care product) applied to the skin. For example, a distal surface of the waveguide is placed in contact with a relatively thin layer (from about 1 mm to about 3 mm, or less) of skin care product that has been applied to the skin. Alternatively, the thin layer can be applied to the distal face of the waveguide, and then the waveguide placed on the skin. The custom cosmetic product is formulated such that when ultrasound energy is directed into the custom cosmetic product via the waveguide, bubbles in the custom cosmetic product oscillate, and this oscillatory motion increases the permeability of the skin to active agents incorporated into the custom cosmetic product. Exemplary liquids and transducer power outputs are discussed in more detail below.

Significantly, the waveguide directs the acoustic energy to the boundary region between the skin care product and the skin. Other products have attempted to focus ultrasound energy to sub-dermal regions, so that the ultrasound energy would have a therapeutic effect on sub dermal tissue. In the context of the present invention, the ultrasound energy is instead directed into the skin care product at the boundary between the applicator and the skin, so that oscillations in the skin care product increase the permeability of the skin, allowing one of more active ingredients in the skin care product to reach sub dermal tissue. In general, the oscillations open up existing pores.

In at least one exemplary embodiment, the acoustic impedance of the skin care product is selected to enable some of the acoustic energy to pass through the skin care product and into the skin to a depth of about 3.5 mm. The purpose for introducing some acoustic energy into the upper dermal tissue (i.e., about the first 3.5 mm) is not to heat the dermal tissue, or for the acoustic energy to have some physiological effect on that tissue. Rather, the acoustic energy, delivered as a wave or pulse, acts as a driving force that pushes some of the skin care product through the pores that have been opened by the oscillating bubble action in the skin care product. Furthermore, the acoustic energy will also generate shear stresses at the skin layer boundary, further facilitating the absorption of the skin care product.

In at least one exemplary embodiment, the acoustic source and waveguide provide sufficient acoustic energy to cause microbubbles to form in the skin care product applied to the skin, and those newly formed microbubbles oscillate to increase the skin permeability. Alternatively, custom formulations of skin care products will include microbubbles or microspheres in addition to the active ingredients. In such embodiments, relatively less acoustic energy is required to cause the microbubbles or microspheres to oscillate and increase skin permeability.

Custom formulations of skin care products can include various active ingredients (generally moisturizers, conditioners, emollients, and/or nutrients, although such ingredients are exemplary, rather than limiting). Preferably, the custom formulations will include either microspheres or microbubbles that can be oscillated, or ingredients that will form such microbubbles when exposed to acoustic energy. In some embodiments, custom formulations of skin care products will also include ingredients whose function is to acoustically match the skin care product to the acoustic energy being employed, to ensure that the acoustical energy will be efficiently absorbed by the skin care product, and that the desired oscillations will occur. Ingredients that can be used to manipulate the acoustical properties of the formulations include (but are not limited to) gelatin, polyoxymethylene urea (PMU), methoxymethyl methylol melamine (MMM), hollow phenolic beads, solid microspheres (spherical styrene/acrylic beads), and calcium aluminum borosilicate (another type of microsphere). It should be noted that some of the above materials are available as hollow microbubbles or solid spheres and either is usable in the present application. An exemplary, but not limiting size range for such spheres/microbubbles is between about 100 nm to about 100 microns. An exemplary, but not limiting concentration of spheres/microbubbles introduced into the skin care product is about 0.2%. The spheres/microbubbles are added for two primary purposes: to change the acoustic properties of the skin care product, to ensure that the skin care product absorbs acoustic energy as much as practical; and, to increase the permeability of the skin due to the oscillation of the spheres/microbubbles.

In some exemplary embodiments, the waveguide is incorporated into a removable therapy head (e.g., where the waveguide is included in the therapy head). Of course, an integrated device with no removable components can also be provided for this application.

In some exemplary embodiments, a motor is configured to energize a vibrational structure at sonic frequencies. Exemplary vibrational elements include conformal pads, bristles, or the therapy head itself. The vibrational element is not required, but may provide a more pleasant user experience. In at least some embodiment, the motor will be controlled to provide pulsations (i.e., motor frequencies) ranging from about 5 kHz to about 10 kHz.

In at least one exemplary embodiment, no bristles or other elements extend beyond the distal face of the acoustic wave guide, which would contact the user's skin while the applicator is in use.

This Summary has been provided to introduce a few concepts in a simplified form that are further described in detail below in the Description. However, this Summary is not intended to identify key or essential features of the claimed subject matter, nor is it intended to be used as an aid in determining the scope of the claimed subject matter.

### DRAWINGS

Various aspects and attendant advantages of one or more exemplary embodiments and modifications thereto will become more readily appreciated as the same becomes better understood by reference to the following detailed description, when taken in conjunction with the accompanying drawings, wherein:

FIG. 1 is an exploded view of the basic elements used in an exemplary cosmetic therapy devices in accord with the concepts disclosed herein, illustrating details to show how acoustic energy is focused at a skin care product disposed between the distal face of the acoustic waveguide and the skin, such that the acoustic energy causes microbubbles in the skin care product to oscillate, to enable an active ingredient in the skin care product to penetrate a stratum corneum layer of the skin, such that the active ingredient is delivered to sub dermal tissue to improve skin quality;

FIG. 2 schematically illustrates an exemplary waveguide for focusing acoustic energy at the skin care product disposed between the distal face of the acoustic waveguide and the skin;

FIG. 3 schematically illustrates how components of an exemplary waveguide for focusing acoustic energy at the skin care product disposed between the distal face of the acoustic waveguide and the skin can be tuned to optimize transmission of the acoustic energy into the skin care product;

FIG. 4 schematically illustrates an exemplary applicator that uses a waveguide to focus acoustic energy at the skin care product disposed between the distal face of the acoustic waveguide and the skin, such that the acoustic energy causes microbubbles in the skin care product to oscillate, such the oscillation enables an active ingredient in the skin care product to penetrate a stratum corneum layer of the skin, such that the active ingredient is delivered to sub dermal tissue to improve skin quality;

FIG. 5 is an exploded view of the exemplary applicator of FIG. 4;

FIG. 6A schematically illustrates a triangular form factor for a therapy head including an acoustic waveguide and a single acoustic transducer for the exemplary applicator of FIG. 4;

FIG. 6B schematically illustrates a triangular form factor for a therapy head including an acoustic waveguide and a plurality of acoustic transducers for the exemplary applicator of FIG. 4;

FIG. 6C is an exploded view of an acoustic transducer and waveguide for the exemplary applicator of FIG. 4;

FIGS. 7A and 7B schematically illustrate exemplary distal surfaces for the acoustic waveguide in the exemplary therapy head of FIG. 6A;

FIGS. 8A and 8B schematically illustrate details of an exemplary removable therapy head including an acoustic waveguide and an acoustic transducer for the exemplary applicator of FIG. 4;

FIG. 9 schematically illustrates a radial transducer and transducer housing for the exemplary applicator of FIG. 4;

FIG. 10 schematically illustrates an alternative transducer design for the exemplary applicator of FIG. 4; and

FIGS. 11A-11F schematically illustrate exemplary alternative designs for the distal surface of the acoustic waveguide for various applicators disclosed herein.

### DESCRIPTION

### Figures and Disclosed Embodiments are not Limiting

Exemplary embodiments are illustrated in referenced Figures of the drawings. It is intended that the embodiments and Figures disclosed herein are to be considered illustrative rather than restrictive. No limitation on the scope of the technology and of the claims that follow is to be imputed to the examples shown in the drawings and discussed herein.

In an exemplary embodiment, the acoustic energy employed has a frequency ranging from about 100 kHz to about 500 kHz. In a first related exemplary embodiment, the acoustic energy employed has a frequency ranging from about 300 kHz to about 350 kHz. In a second related exemplary embodiment, the acoustic energy employed has a frequency ranging from about 200 kHz to about 250 kHz. In general, the term ultrasound is employed to refer to sound of a frequency higher than about 20 kHz (i.e., sound outside of the audible range of the human ear). The term acoustic energy encompasses ultrasound, as well as encompassing frequencies not generally referred to as ultrasound.

The concepts disclosed herein utilize ultrasound waveguide technology and sonic vibrations to provide deeper penetration of therapeutic chemicals, such as cleansing and anti-aging products. More particularly, these concepts provide a non-invasive method of compound delivery through the epidermis by means of increasing the permeability of the skin through small hydrophilic channels in the stratum corneum. The channels are naturally occurring, and they become enlarged due to the oscillations.

The human skin has barrier properties, and the stratum corneum (the outer horny layer of the skin), is mostly responsible for these barrier properties. The stratum corneum imposes the greatest barrier to the transcutaneous flux of compounds into the body and is a complex structure of compact keratinized cell remnants separated by lipid domains. It is formed from keratinocytes, which comprise the majority of epidermal cells that lose their nuclei and become comeocytes. These dead cells make up the stratum corneum, which has a thickness of only about 10-30 gm, and which provides a waterproof membrane that protects the body from invasion by external substances, as well as preventing the outward migration of fluids and dissolved molecules.

Traditional applications of creams and lotions just sit on the surface of the skin. Using the concepts disclosed herein, skin care products can now penetrate the skin's surface and go to work to produce visible, desired results. Not only will the skin be extremely clean and rejuvenated (as a result of acoustic scrubbing of the skin surface), the micro-rubbing action will also tighten the skin's surface for a more youthful, toned appearance.

In prior art ultrasonic-based skin treatment devices, a probe is used to apply ultrasonic vibrations to the area of cosmetics application; however, the ultrasonic waves propagate along the skin line or penetrate into a sub dermal layer. Significantly, such prior art devices do not focus the acoustic energy at skin care product disposed between the acoustic applicator and the skin, such that the acoustic energy causes microbubbles in the skin care product to form and/or oscillate. As discussed above, such oscillation enables an active ingredient in the skin care product to penetrate a stratum corneum layer of the skin, such that the active ingredient is delivered to sub dermal tissue to improve skin quality.

The cosmetic treatment devices disclosed herein generally include an acoustic waveguide, and an ultrasound transducer assembly. Some exemplary embodiments include a drive motor for vibrating the therapy head to provide a massage effect (though such vibrations are not a major component of inducing the micro bubble oscillations required to improve skin permeability). The acoustic energy generates bubbly flow and shear stresses at the tissue boundary and improves penetration of the active ingredient across the skin barrier. The combination of the ultrasound transducer and acoustic waveguide focusing the acoustic energy into the skin care product provide an effective cosmetic treatment device, yielding a synergistic treatment effect in combination with the active ingredients in the skin care product.

Skin active agents (i.e., therapeutic agents or active ingredients) to be used in conjunction with the described acoustic applicator can include (individually or in combination):
Nanosphere technology—infused with free-radical fighting antioxidant vitamins, which can penetrate deep into the skin (once past the skin barrier) to protect, condition, and adjust to the skin's specific needs;
Oil-Free agents (no occlusive mineral oils or lanolins);
GABA (gamma amino butyric acid)—which may reduce the muscle movements partly responsible for the expression of wrinkles;
Niacinamide—which prompts increased synthesis of collagen and keratin, decreases UV-induced skin cancers, and helps decrease facial pigmentation. and which brightens dull and sallow skin;
Coenzyme Q10—which may boost skin repair and regeneration and reduce free radical damage (a small molecule that can relatively easily penetrate into skin cells), once past the skin barrier;
Peptides—which reduce the skin's roughness and also reduces the appearance of wrinkle depth and volume;
Antioxidants—which keep the skin healthy by fighting free-radical damage;
Hyaluronic Acid—which holds 100 times its weight in water (i.e., it is a great hydrator);
DMAE—which can help tighten sagging skin;
Alpha lipoid acid—which is a powerful antioxidant that penetrates skin quickly and absorbs into the skin's cells to increase metabolism;
Vitamin C Ester—which promotes collagen, elastin, and ground substance (the strength and elasticity of the skin);
Green/white tea extract—which includes naturally occurring anti-oxidants;
Kojic Acid—which is a natural skin lightening agent that reduces the appearance resulting from long-term sun exposure and environmental damage;
Alpha and Beta Hydroxy Acids—which activate healthy cells, while diminishing the appearance of fine lines and wrinkles; and
Phytoestrogens.

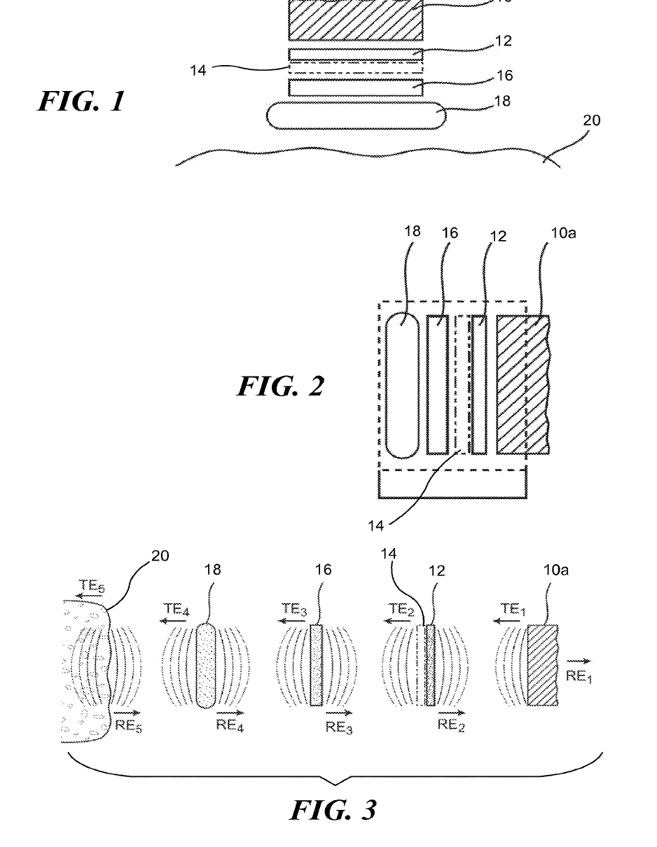

FIGS. 1-11F refer to an exemplary applicator. It should be recognized that this applicator is not limiting on the concepts disclosed herein. For example, different applicators having different form factors are encompassed by the concepts disclosed herein. Also encompassed by the concepts disclosed herein are different transducer designs. Furthermore, while the exemplary applicator employs a battery power source, it should be recognized that the battery can be replaced by a power cord to be plugged into a conventional electrical outlet or even an accessory power outlet in a vehicle.

FIG. 1 is an exploded view of the basic elements used in cosmetic therapy devices in accord with the concepts disclosed herein, providing details on how acoustic energy is focused at the boundary between a skin care product and the skin, such that the acoustic energy causes microbubbles in the skin care product to oscillate, so that the oscillation enables an active ingredient in the skin care product to penetrate a stratum corneum layer of the skin, delivering the active ingredient to sub dermal tissue to improve skin quality. Note that while the elements are shown as being spaced apart in this exploded view, when in use, adjacent waveguide elements will be in contact with each other (or separated by a thin layer of adhesive having mechanical properties selected such that the thin layer does not negatively affect the acoustic properties of the waveguide).

Referring to FIG. 1, an acoustic transducer 10 (in an exemplary, but not limiting embodiment the acoustic transducer is an ultrasound transducer) is coupled to one or more matching layers (i.e., matching layers 12 and 14), a skin contact layer 16 (referred to elsewhere as the distal face of the waveguide), and a skin care product 18 that is applied to a skin surface 20. In general, the skin care product is first applied to the skin, but in at least one embodiment the skin care product is first applied to the distal face of the waveguide.

It should be noted that while the waveguide is configured to direct acoustic energy into the skin care product disposed between the applicator and the skin, it is advantageous for the acoustic impedance of the skin care product to enable some of the acoustic energy to pass through the skin care product and into the skin to a depth of about 3.5 mm. The purpose for introducing some acoustic energy into the upper dermal tissue (i.e., about the first 3.5 mm) is not to heat the dermal tissue, or for the acoustic energy to have some physiological effect on that tissue. Rather, the acoustic energy, delivered as a wave or pulse, acts as a driving force that pushes some of the skin care product through the pores that have been opened by the oscillating bubble action in the skin care product. Furthermore, the acoustic energy will also generate shear stresses at the skin layer boundary, further facilitating the absorption of the skin care product.

FIG. 2 schematically illustrates an exemplary waveguide for focusing acoustic energy at the skin care product disposed between the distal face of the acoustic waveguide and the skin care product. A PZT ceramic transducer 10a is coupled to one or more matching layers (i.e., matching layers 12 and 14). The distal most matching layer is coupled to skin contact layer 16 (i.e., the layer defining the distal face of the waveguide). Skin care product 18 is applied to skin surface 20, generally as discussed above. Collectively, the matching layers and the skin contact layer define a waveguide directing acoustic energy into the skin care product.

FIG. 3 schematically illustrates how exemplary components of a waveguide for focusing acoustic energy at the skin care product disposed between the distal face of the acoustic waveguide and the skin can be tuned to optimize transmission of the acoustic energy into the skin care product. In order to achieve such tuning, the acoustic impedance of each material is selected to maximize the ultrasound transmission into the subsequent layer, while minimizing the reflected acoustic energy. In this Figure, this tuning can be seen as the transmitted ultrasound energy (TEI) from PZT ceramic transducer I0a propagates to matching layer 12 and matching layer 14, skin contact layer 16, and skin care product 18. Each component of the waveguide is designed (via the addition of certain chemical or mechanical enhancers) to have an acoustic impedance that maximizes the transmitted ultrasound energy (TE1-TE5), while minimizing the reflected energy (RE1-RE5). More specifically, this description pertains to the acoustic impedance of the skin care product 18, which must be able to absorb sufficient acoustic energy to induce the micro bubble oscillations that increase the skin permeability. In at least one embodiment, some amount of the acoustic energy will pass through the skin care product into the skin (as indicated by TE5) to provide a flux to drive the active ingredient of the skin care product through the openings formed in the skin by the microbubble oscillations. Thus, the acoustic impedance of each layer between the transducer and the skin is selected to maximize the transmitted acoustic energy into the skin care product.

FIG. 4 schematically illustrates an exemplary applicator 22 that uses a waveguide to focus acoustic energy at the skin care product disposed between a distal face of the waveguide and the skin, such that the acoustic energy causes microbubbles in the skin care product to oscillate, such oscillation enabling an active ingredient in the skin care product to penetrate a stratum corneum layer of the skin, such that the active ingredient is delivered to sub dermal tissue to improve skin quality. Applicator 22 includes an outer casing, within which is disposed a rechargeable battery 36 (such as a lithium ion or other rechargeable battery) that provides electrical power to a timing controller 34, an electrical drive circuit 30, a vibration motor 28, and acoustic transducer 10. Timing controller 34 provides timing, motor control, and various control functions for the applicator and is connected to electrical drive circuit 30, which includes an acoustic module drive circuit to provide the necessary electrical drive to the acoustic transducer. Electrical drive circuit 30 is further connected to a motor drive 32, which provides electrical power to motor 28. Motor 28 is not strictly required, and is provided to vibrate the therapy head (i.e., the transducer and the waveguide) to provide a pleasant massaging effect. Further, vibrating the therapy head can help disperse the skin care product on the skin. Electrical drive circuit 30 is further connected to electrical contacts 24, which connect to a removable transducer housing 26, providing electrical contact between transducer 10 and electrical drive circuit 30. Transducer housing 26 contains the ultrasound transducer and waveguide, and is connected to electrical drive circuit 30 via electrical contacts 24. Generally as discussed above, a waveguide 38 includes multiple layers, including matching layers 12 and 14, and skin contact layer 16, to acoustically couple skin care product 18 to transducer 10, to focus the acoustic energy into the skin care product.

Including a plurality of matching layers in the waveguide has an advantage. When an acoustic wave encounters a boundary between two layers having a relatively large variance in their respective acoustic impedances, the acoustic wave is reflected at the boundary. Using a plurality of layers enables the acoustic impedence of each layer to be varied gradually, to minimize reflections. The larger the difference in the acoustic impedances of the skin care product and the acoustic source, the more matching layers should be employed to minimize reflections. In at least one embodiment, the acoustic impedance of the skin care product is matched closely enough to the acoustic impedance of the skin boundary, such that reflections at the skin layer boundary are minimized. As noted above, it is desirable to have some of the acoustic energy pass through the skin layer boundary, into the tissue to a depth of about 3.5 mm, to provide a force that pushes the skin care product through the pores opened by the oscillating motion in the skin care product. In other words, the matching layers in the acoustic waveguide directs acoustic energy from the transducer to the skin care product, and the skin care product acts as a matching layer/waveguide to direct some of the ultrasound into the upper layers of the dermal tissue.

FIG. 5 is an exploded view of the exemplary applicator of FIG. 4. Note that the housing includes an upper shell 40 and a bottom shell 42. The applicator includes a removable transducer housing 44, in which are disposed transducer 10 and waveguide 38. Disposed within the elongate housing (i.e., shells 40 and 42) are a battery charging unit 45, a battery charging connector 46, and a battery 48. In an exemplary, but not limiting embodiment the battery charging unit is based on induction (it should also be noted that the concepts disclosed herein further encompass applicators alternatively powered by removable batteries, or applicators with a power cord enabling the applicators to be coupled to a power source, such as a conventional electrical outlet). Battery charging connector 46 connects battery 48 to battery charging unit 45. A printed circuit board 50 is also disposed in the elongate housing, along with a transducer receiver 52, which releasably engages removable transducer housing 44.

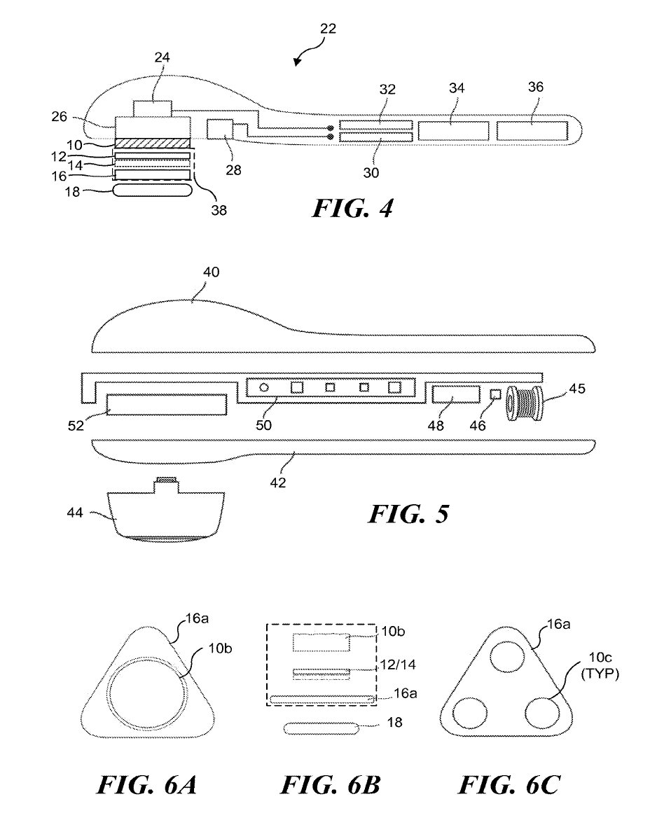

FIG. 6A schematically illustrates a triangular form factor for a removable therapy head including an acoustic waveguide and a single acoustic transducer 10b for the exemplary applicator of FIG. 4. The triangular shape (with rounded corners) enables coverage of hard to reach places on the face during operation of the applicator, particularly near the eyes and nose. FIG. 6B is an exploded view of the removable therapy head of FIG. 6A, which includes acoustic transducer 10b and a waveguide for the exemplary applicator of FIG. 4. Transducer 10b is connected to matching layers 12 and 14, and then to the skin contact layer 16a. Significantly, it is the skin contact layer that exhibits the triangular form factor. As discussed above, the one or more matching layers and the skin contact layer collectively comprise the waveguide, such that the lower surface of the skin contacting layer is the distal face of the waveguide. Note that in FIG. 6B, the elements in the removable therapy head are shown in a dashed box. Skin care product 18 is not part of the removable therapy head, but is also shown to indicate how the removable therapy head is used. FIG. 6C schematically illustrates a triangular form factor for a removable therapy head including an acoustic waveguide and a plurality of acoustic transducers 10c.

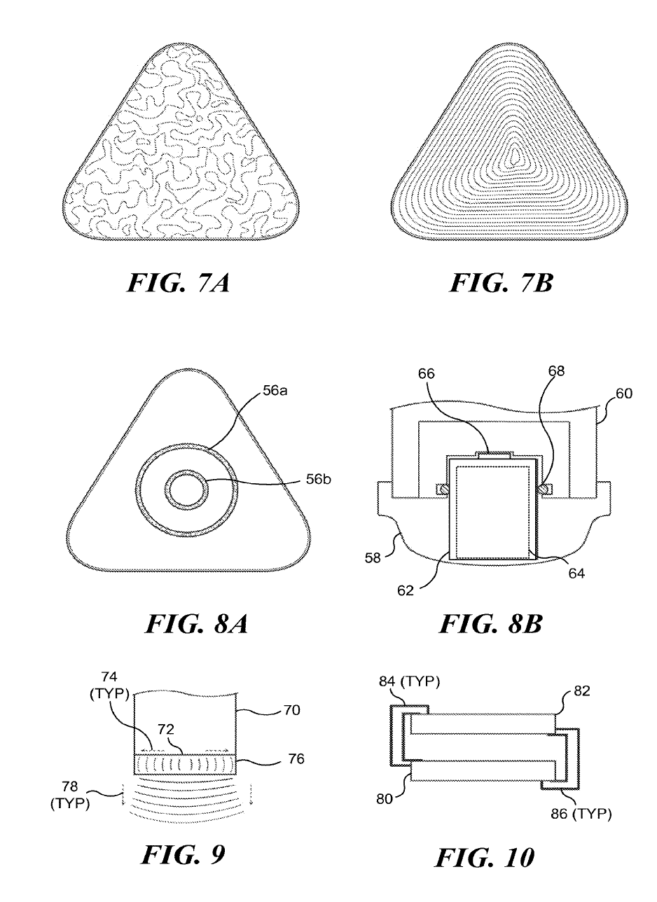

FIGS. 7A and 7B schematically illustrate exemplary distal surfaces for the acoustic waveguide in the exemplary therapy head of FIG. 6A. The distal surface of skin contact layer 16a can be implemented in a variety of ways. Referring to both the surface designs of FIGS. 7A and 7B, the designs are beneficially implemented using materials mimicking the feel and durometer of human skin, while maintaining the desired acoustic impedance for the waveguide.

FIGS. 8A and 8B schematically illustrate details of an exemplary removable therapy head including an acoustic waveguide, and an acoustic transducer for the exemplary applicator of FIG. 4. Referring to FIG. 8A, a top view of the removable therapy head of FIG. 6A or 6C shows electrical contacts 56a and 56b to electrically couple the transducer(s) in the removable therapy head to the driving components in the elongate housing of the applicator (see FIGS. 4 and 5 for details of the driving electronics and power supply). Electrical contacts 56a and 56b are designed as concentric rings, with a ground contact 56a as the outer ring and a signal line contact 56b as the inner ring. In this embodiment electrical connection can be made regardless of how the removable transducer housing/therapy head is oriented during installation.

FIG. 8B schematically illustrates a removable transducer housing/therapy head 58 being attached to a handle 60 (note an elongate handle including the driving electronics, control electronics, and power supply are generally described above in connection with FIGS. 4 and 5). Removable therapy head 58 includes a transducer housing 62, which itself includes the acoustic transducer and waveguide matching layers discussed above (such elements are generally indicated as element 64). The skin contact layer discussed above forms the outer shell of the removable transducer housing. Removable therapy head 58 is inserted into a receiver portion 66 of handle 60, and a seal 68 (such as an O-ring or functional equivalent) prevents water from leaking into housing 60.

Acoustic transducers are often designed to function in a longitudinal mode. FIG. 9 schematically illustrates an exemplary radial transducer and transducer housing embodiment. In such an embodiment, the transducer housing is designed so that the PZT ceramic can be operated in a radial mode. A transducer housing 70 secures a transducer 72, operated in the radial mode as indicated by arrows 74. A portion of the housing proximate to the transducer provides a contact barrier 76 on the outer portion of the radially oriented transducer. This contact barrier converts the radial mode into a longitudinal mode of operation during use, as indicated by arrows 78. Under certain drive conditions, the radial mode enables the acoustic output to exhibit a plurality of acoustic frequencies.

FIG. 10 schematically illustrates another alternative transducer design for the exemplary applicator of FIG. 4, in which dual longitudinally operated transducers are used in parallel. In such an embodiment, the transducer housing (not separately shown) includes two longitudinal transducers connected in parallel. A first transducer 80 is connected to second transducer 82 in parallel using signal lines 84 and ground lines 86. Such an embodiment reduces the electrical voltage required to drive the transducer component, thereby reducing the size of the drive circuitry in the handle.

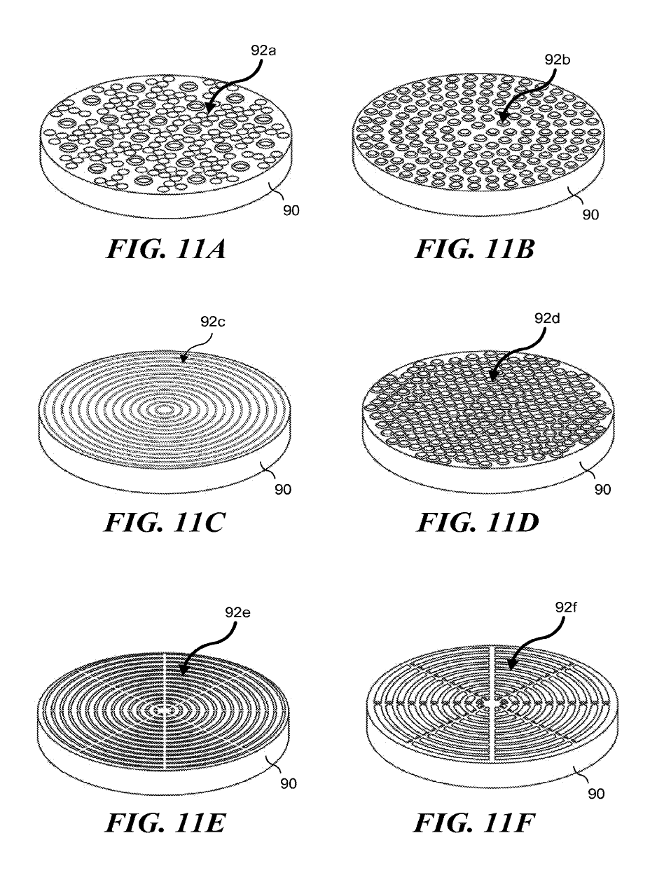

FIGS. 11A-11F schematically illustrate alternative designs for the distal surface of the acoustic waveguide for various applicators disclosed herein. As noted above, the distal surface is also referred to herein as the skin contact layer and is the external surface of the waveguide. The form factors shown in FIGS. 11A-11F are circular, although it should be understood that such a form factor is exemplary and not limiting.

Referring to FIGS. 11A-11F, it should be understood that each body 90 is a layer in the acoustic waveguide, and thus each body 90 is formed out of a material that ensures that the acoustic energy from the acoustic transducer is focused on the skin care product immediately adjacent to each distal surface 92a-92f. As discussed above, the skin care product is applied to the skin (or to the distal surface itself), such that the skin care product is disposed between the distal surface and the skin. The acoustic energy directed into the skin care product causes hollow bubbles or solid microspheres already present in the in skin care product (or hollow bubbles formed in the skin care product in response to the absorption of the acoustic energy) to oscillate and increase the permeability of the skin. Because the distal surface will be very close to the user's skin (separated only by a relatively thin layer of the skin care product), various surface features can be included in the distal surface to enhance user satisfaction with the applicator. In generally, the distal surface should not generate unpleasant sensations when the distal surface touches the skin. The durometer of the distal surface can range from about 75 Shore A to 20 Shore A, with a particularly desired durometer being about 40 Shore A (i.e., about the same as a human fingertip). While many materials can be used to implement each distal surface, silicone compositions are particularly suitable.

FIGS. 11A-11F schematically illustrate different types of distal surfaces, each including different surfaces features (note that such surface features can be implemented as either depressions or protrusions). A distal surface 92a of FIG. 11A includes a plurality of generally circular surface features (which vary in size), distributed in a random pattern. A distal surface 92b of FIG. 11B also includes a plurality of generally circular surface features, however these surface features are distributed in an ordered pattern of concentric rings, each ring including a plurality of circular surface features. A distal surface 92c of FIG. 11C also includes an ordered pattern of concentric rings, however here each ring is defined by a contiguous surface feature (as opposed to each ring being defined by a plurality of circles). A distal surface 92d of FIG. 11D also includes an ordered pattern including a plurality of generally circular surface features, however here the circles are arranged in a two dimensional linear array. A distal surface 92e of FIG. 11E also includes an ordered pattern of concentric rings, however here the rings are separated into a plurality of equal sized sectors. A distal surface 92f of FIG. 11F is similar to distal surface 92e of FIG. 11E, however each concentric ring feature is relatively thicker in distal surface 92f.

The exemplary applicator discussed above represents just one of many possible applicator embodiments. The following provides a brief discussion of other applicators and embodiments, consistent with the concepts disclosed herein.

In one exemplary, but not limiting embodiment, the skin care device includes: (1) a single applicator handle having a pulsed acoustic generator and a motor coupled to the support structure, which together provide electrical and mechanical signals to a removable therapy contact; and, (2) at least one removable therapy head. Useful removable therapy heads include: a removable therapy head having an acoustic waveguide in the center surrounded by at least one ring of bristles, each bristle being coupled to a ring connected to the removable therapy head, each ring being configured to rotate upon connection to the motor drive; and, a removable skin care therapy head having an acoustic waveguide in the center, surrounded by a soft conformable pad that forms a pocket when contacting the skin surface, the conformable pad being connected to the removable head contact and providing pulsation when coupled to an driven by the motor drive.

An exemplary acoustic transducer for use in one or more of the embodiments disclosed herein produces ultrasonic energy at frequencies between 25 KHz and 500 KHz, generating a peak negative acoustic pressure of about 0.1-1 MPa during a single acoustic cycle.

In some applicator embodiments in which a portion of the therapy head is configured to vibrate or rotate, exemplary vibration/rotation parameters include a peak velocity less than 3 m/sec, and a motor frequency 10 kHz.

In some exemplary embodiments, the acoustic waveguide is mounted to and contacts the upper surface of the transducer, and at least a portion of the side walls of the transducer.

In some exemplary embodiments, the acoustic transducer operates in a pulsed mode where the pulse frequency is not greater than 2 KHz. The acoustic transducer generates sinusoidal acoustic waves that operate at an ultrasonic energy at frequencies of less than 500 KHz, and produces a peak negative acoustic pressure between 0.1-1 MPa during one acoustic cycle. The total average power of the acoustic output need not exceed 0.25 mW.

In some exemplary embodiments, the acoustic transducer includes at least one piezoelectric element.

In some exemplary embodiments, the acoustic transducer includes a flat, circular piezoelectric element.

In some exemplary embodiments, the acoustic transducer includes a series of piezoelectric elements arranged in a circular array so that their acoustic emission combines at a natural geometric focus.

In some exemplary embodiments, the acoustic transducer includes a stack of piezoelectric elements.

In some exemplary embodiments, the acoustic transducer includes a series of piezoelectric elements arranged in a triangular array so that their acoustic emission combines at a natural geometric focus.

In some exemplary embodiments, the acoustic transducer includes a piezoelectric element having electrically conductive material on one side of its surfaces.

In some exemplary embodiments, the acoustic transducer includes a piezoelectric element having acoustically matched material connected to the waveguide.

In some exemplary embodiments, the acoustic transducer operates to produce ultrasonic energy at frequencies of less than 250 KHz during an acoustic cycle.

In some exemplary embodiments, the acoustic transducer is pulsed at a pulse frequency of no more than 2 KHz.

In some exemplary embodiments, the acoustic transducer operates at no more than 0.25 mW average power.

In some exemplary embodiments where a motor is used to vibrate or rotate a portion of the therapy head, the motor operates to rotate and or vibrate the portion at a peak velocity of less than 2 m/sec during one cycle.

In some exemplary embodiments where a motor is used to vibrate or rotate a portion of the therapy head, the motor operates to rotate and or vibrate the portion at a frequency of less than 250 KHz.

In some exemplary embodiments, an ultrasound drive circuit is mounted in the handle and electrically coupled to an ultrasound piezoelectric element comprising the transducer, wherein the ultrasound drive circuit is controlled by a circuit board, receiving power from a rechargeable battery.

In some exemplary embodiments, a removable therapy head includes an acoustic waveguide disposed in a center of a rotating brush ring.

In some exemplary embodiments, the therapy head and handle are integrated and non removable.

In some exemplary embodiments, the acoustic waveguide is dome shaped.

In some exemplary embodiments, the acoustic waveguide has a flat circular disk shape.

In some exemplary embodiments, the acoustic waveguide has a pyramid shape.

In some exemplary embodiments, the acoustic waveguide has a flat circular spiral shape.

In some exemplary embodiments, the acoustic waveguide has a flat square shape.

In some exemplary embodiments, the acoustic waveguide has a triangular shape.

In some exemplary embodiments, the acoustic waveguide is made from a non stick material.

In some exemplary embodiments, the acoustic waveguide is made from a silicon material.

In some exemplary embodiments, the acoustic waveguide is made from a material acoustically matched to human skin.

In some exemplary embodiments, the acoustic waveguide is made from a material acoustically matched to the acoustic transducer.

In some exemplary embodiments, the acoustic waveguide is made from a material acoustically matched to both the acoustic transducer and human skin.

In some exemplary embodiments, the therapy head includes a rotating brush ring having a set of soft bristles made from nylon or plastic.

In some exemplary embodiments, the therapy head includes a rotating brush ring having a set of soft bristles made from a soft material suitable for skin contact.

In some exemplary embodiments, the therapy head includes an acoustic waveguide in the center of a conformable vibrating pad. In such an embodiment, the conformable pad material can be made from a soft, conformable material suitable for skin contact. In at least some related embodiments, the conformable pad provides a 23 mm standoff between the skin surface and the waveguide.

In some exemplary embodiments, the transducer generated acoustic energy in combination with the skin care product results in acoustic cavitation on the surface of the skin. In at least some related embodiments, the acoustic cavitation produces shear stress on the skin surface.

In some exemplary embodiments, the transducer generated acoustic energy in combination with the skin care product results in acoustic cavitation in the skin care product. In at least some related embodiments, the acoustic cavitation produces acoustic streaming in the skin care product.

In some exemplary embodiments, the transducer generated acoustic energy in combination with the skin care product results in stable cavitation on the surface of the skin. In at least some related embodiments, the stable cavitation produces shear stress on the skin surface.

In some exemplary embodiments, the transducer generated acoustic energy in combination with the skin care product results in stable cavitation in the skin care product. In at least some related embodiments, the stable cavitation produces acoustic streaming in the skin care product.

In some exemplary embodiments, the transducer generated acoustic energy in combination with the skin care product results in stable bubble oscillations on the skin surface. In at least some related embodiments, the stable bubble oscillations on the skin surface produce shear stress on the skin surface.

In some exemplary embodiments, the transducer generated acoustic energy in combination with the skin care product results in stable bubble oscillations in the skin care product. In at least some related embodiments, the stable bubble oscillations in the skin care product produce acoustic streaming in the skin care product.

In some exemplary embodiments, the transducer generated acoustic energy in combination with the skin care product generates bubbles in the skin care product or on the skin surface.

An exemplary method consistent with the concepts disclosed herein includes the steps of: (1) providing a safe and therapeutically effective amount of a composition including a skin active agent, the composition having a viscosity ranging from about 500-5000 mPA when measured with a Brookfield rotational viscometer, the composition having from about 0.5 to about 20 parts by weight of water-soluble humectants or a nonionic surfactant, and an aqueous carrier, and/or an absorption activator (benzyl alcohol, sodium laurel sulfate, etc.); and, (2) applying ultrasound to the surface of the skin by an ultrasound applying apparatus. The ultrasound applying apparatus preferably includes an application element for applying ultrasound at a frequency of from about 25 KHz and 500 kHz to the skin, where the total average power of the acoustic output need not be more than 0.25 mW and a control element for controlling application conditions of the application element. In such a method, the composition is used as a medium for applying ultrasound to the skin by the ultrasound applying apparatus.

In at least one related method, the composition is formulated with at least one chemical designed to enhance bubble formation by ultrasound energy.

In at least one related method, the composition is formulated with at least one chemical designed to enhance the production of sheer stress on the skin surface by ultrasound energy.

In at least one related method, the composition is formulated with at least one chemical designed to enhance the production of acoustic streaming in the composition by ultrasound energy.

An exemplary (but not limiting) skin therapy system includes an ultrasonic transducer acoustically coupled to a skin care product applied to human skin through the use of an acoustic waveguide. The acoustic waveguide includes one or more matching layers designed to focus the acoustic energy into the skin care product applied to human skin. The acoustic properties of the waveguide are designed to maximize acoustic absorbance in the skin care product applied to human skin, by matching the impedance of the transducer, acoustic waveguide, and the skin care product. The acoustic energy enhances the absorption of at least one of the active ingredients of the skin care product into the skin.

An exemplary waveguide for such a system has an acoustic impedance of about 0.5-3.5 MRayl's in a frequency range of about 100 KHz-2 MHz. Upon propagation through the waveguide, the acoustic intensity of ultrasonic energy in the skin care product applied to human skin is in the range of about 0.1 W/cm²-1 W/cm².

Another exemplary waveguide for such a system has an acoustic impedance of about 0.5-3.5 MRayl's in a frequency range of about 100 KHz-2 MHz. Upon propagation through the waveguide, the acoustic intensity of ultrasonic energy in the skin care product applied to human skin is in the range of about 0.01 W/cm²−1 W/cm².

An exemplary skin care device consistent with the concepts disclosed herein includes a single applicator handle in which are disposed a pulsed acoustic generator and a motor coupled to a support structure, which together provide electrical and mechanical signals to a removable and interchangeable therapy head contact.

Such an exemplary skin care device can include an acoustic transducer acoustically coupled to an acoustic waveguide that produces ultrasonic energy at frequencies in the range from about 100 kHz to about 2 MHz, producing peak negative acoustic pressures of about 0.1-1 MPa during one acoustic cycle.

Such an exemplary skin care device can include a removable and interchangeable skin care therapy head having an acoustic waveguide surrounded by a soft conformable pad that forms a pocket when contacting the skin surface. The conformable pad is connected to the removable and interchangeable head contact and provides vibration upon being drivingly driven by the motor drive. In at least one related embodiment, the soft conformable pad exhibits the following properties: a durometer ranging from 75 Shore A to 20 Shore A, with a particularly desired durometer being about 40 Shore A. Physical properties of exemplary silicone coverings are as follows: Durometer 40 Shore A; Tensile Strength 800 lb/in²; Elongation 220%; and, Temperature Resistance 400° F. constant.

Such an exemplary skin care device can include brushes and/or one or more conformable pads included in the therapy head portion, such elements being coupled to the support structure via the removable and interchangeable head contact, which connects them to the motor drive. In operation, the peak vibration motor frequency will be 10 kHz and the peak velocity will be less than 3 m/second.

Such an exemplary skin care device can include an acoustic waveguide mounted to and contacting an upper surface of the transducer and at least a portion of the side walls of the transducer.

Such an exemplary skin care device can include an acoustic transducer including at least one piezoelectric element operating in a pulsed mode, where the pulse frequency is not greater than about 2 kHz. In at least one related embodiment, the acoustic transducer generates an acoustic waveform that operates at an ultrasonic energy at frequencies of less than 2 MHz and produces a peak negative acoustic pressure between about 0.1-1 MPa during one acoustic cycle, with the total average power of the acoustic output being less than about 0.25 mW.

Such an exemplary skin care device can include an acoustic transducer based on a series of piezoelectric elements arranged in an array so that their acoustic emission combines at a natural geometric focus.

Such an exemplary skin care device can include an acoustic transducer based on a stack of individual piezoelectric elements.

Such an exemplary skin care device can include an acoustic transducer based on a series of piezoelectric elements driven in a radial mode.

Such an exemplary skin care device can include an acoustic transducer based on a single piezoelectric element driven in a radial mode.

Such an exemplary skin care device can include an acoustic transducer operated to produce ultrasonic energy at frequencies of less than about 2 MHz during an acoustic cycle.

Such an exemplary skin care device can include an acoustic transducer operated to produce ultrasonic energy pulsed at a pulse frequency less than 2 kHz.

Such an exemplary skin care device can include an acoustic transducer operated to produce ultrasonic energy of less than about 0.25 mW average power.

The motor in such an exemplary skin care device can be configured to rotate and or vibrate a portion of the removable head at a peak velocity of less than about 3 m/second during one cycle and at a motor frequency 10 kHz

Such an exemplary skin care device can include an ultrasound drive circuit mounted in the handle and electrically coupled to the ultrasound piezoelectric element comprising the transducer, wherein the ultrasound drive circuit is controlled by a circuit board, receiving power from a rechargeable battery.

An exemplary skin care product is formulated to provide an acoustic impedance matching that of the acoustic transducer and the acoustic waveguide, to enhance the absorption of at least one active ingredient in the skin care product into the skin. Such a skin care product can be a cream, a gel, or a serum.

Such an exemplary skin care product can be formulated to provide an acoustic impedance in the range of about 0.5-3.5 MRayl's.

Such an exemplary skin care product can be formulated with at least one ingredient designed to enhance bubble formation by ultrasound energy.

Such an exemplary skin care product can be formulated with at least one ingredient selected to enhance the production of sheer stress on the skin surface in response to ultrasound energy.

Such an exemplary skin care product can be formulated with at least one type of hollow microbubbles or solid microspheres. Exemplary hollow microbubbles include collagen microbubbles and albumen microbubbles.

Although the concepts disclosed herein have been described in connection with the preferred form of practicing them and modifications thereto, those of ordinary skill in the art will understand that many other modifications can be made thereto within the scope of the claims that follow. Accordingly, it is not intended that the scope of these concepts in any way be limited by the above description, but instead be determined entirely by reference to the claims that follow.

The invention in which an exclusive right is claimed is defined by the following:

1. A method for improving skin quality, comprising the steps of:
(a) providing a skin care product selected to enhance skin quality, the skin care product including an active ingredient for improving skin quality;
(b) applying the skin care product to a portion of skin;
(c) directing acoustic energy at the skin care product applied to the skin, the acoustic energy causing microbubbles in the skin care product to oscillate, the oscillation enabling the active ingredient to penetrate a stratum corneum layer of the skin, so that the active ingredient is delivered to sub dermal tissue to improve skin quality.

2. The method of claim 1, wherein the step of directing acoustic energy at the skin care product applied to the skin comprises the step of using an acoustic waveguide to direct the acoustic energy into the skin care product applied to the skin, thereby reducing an amount of acoustic energy that propagates along a skin boundary layer, the acoustic energy propagating along the skin boundary layer being ineffective with respect to enabling the active ingredient to penetrate a stratum corneum layer of the skin.

3. The method of claim 1, wherein the step of directing acoustic energy at the skin care product applied to the skin comprises the step of using sufficient acoustic energy to generate the microbubbles in the skin care product.

4. The method of claim 1, wherein the step of providing the skin care product selected to enhance skin quality comprises the step of providing a skin care product comprising the microbubbles, an amount of the microbubbles included in the skin care product having been selected to facilitate the oscillation required to enable the active ingredient to penetrate the stratum corneum layer of the skin.

5. The method of claim 1, wherein the step of providing a skin care product selected to enhance skin quality comprises the step of providing a skin care product comprising solid microspheres, an amount of solid microspheres having been selected to acoustically match the skin care product to the acoustic energy directed at the skin care product applied to the skin.

6. The method of claim 1, wherein the step of providing a skin care product selected to enhance skin quality comprises the step of providing a skin care product comprising:
(a) the microbubbles, an amount of the microbubbles included in the skin care product having been selected to facilitate the oscillation required to enable the active ingredient to penetrate the stratum corneum layer of the skin; and
(b) solid microspheres, an amount of solid microspheres having been selected to acoustically match the skin care product to the acoustic energy directed at the skin care product applied to the skin.

7. An applicator for enabling an active ingredient in a skin care product applied to a user's skin to penetrate a stratum corneum layer of the skin; the apparatus comprising:
(a) a handle configured to be grasped by a user to enable the user to selectively position the apparatus proximate to the user's skin;
(b) a therapy head configured to be placed in contact with a skin care product applied to the user's skin, the therapy head including an acoustic waveguide, such that when the apparatus is in use, a distal face of the acoustic waveguide physically contacts a skin care product applied to the user's skin; and
(c) a selectively actuatable acoustic source disposed to direct acoustic energy to the distal face of the acoustic waveguide and into a skin care product applied to the user's skin that is contacting the distal face of the waveguide, the acoustic energy causing microbubbles in the skin care product to oscillate, such oscillation enabling an active ingredient in the skin care product to penetrate a stratum corneum layer of the skin, such that an active ingredient is delivered to sub dermal tissue to improve skin quality.

8. The applicator of claim 7, wherein the applicator does not include any element extending beyond the distal face of the acoustic waveguide that would contact a user's skin while the applicator is in use.

9. The applicator of claim 7, wherein the therapy head does not include any bristles extending beyond the distal face of the acoustic waveguide that would contact the user's skin while the applicator is in use.

10. The applicator of claim 7, further comprising a battery to selectively energize the acoustic source.

11. The applicator of claim 7, wherein the therapy head exhibits a triangular form factor, to facilitate positioning the distal face of the waveguide on skin surfaces having limited accessibility.

12. The applicator of claim 7, wherein the therapy head is removable.

13. The applicator of claim 7, wherein the distal face of the waveguide has a durometer and texture approximating that of human skin.

14. A cosmetic system for improving skin quality, the cosmetic system including a skin care product and an applicator, the skin care product including an active ingredient for improving the quality of skin, the applicator comprising:
(a) a handle configured to be grasped by a user to enable the user to selectively position the apparatus proximate to the user's skin;
(b) a therapy head configured to be placed in contact with the skin care product applied to the user's skin, the therapy head including an acoustic waveguide, such that when the apparatus is in use, a distal face of the acoustic waveguide physically contacts the skin care product applied to the user's skin; and
(c) a selectively actuatable acoustic source disposed to direct acoustic energy to the distal face of the acoustic waveguide and into the skin care product applied to the user's skin that is contacting the distal face of the waveguide, the acoustic energy causing microbubbles in the skin care product to oscillate, wherein oscillation of the microbubbles enables the active ingredient in the skin care product to penetrate a stratum corneum layer of the skin, such that the active ingredient is delivered to sub dermal tissue to improve skin quality.

15. The cosmetic system of claim 14, wherein the skin care product comprises microbubbles, an amount of microbubbles included in the skin care product having been selected to facilitate the oscillation required to enable the active ingredient to penetrate the stratum corneum layer of the skin.

16. The cosmetic system of claim 14, wherein the skin care product comprises solid microspheres, an amount of the solid microspheres included in the skin care product having been selected to acoustically match the skin care product to the acoustic energy directed at the skin care product applied to the skin.

17. The cosmetic system of claim 14, wherein the skin care product comprises:
(a) the microbubbles, an amount of the microbubbles included in the skin care product having been selected to facilitate the oscillation required to enable the active ingredient to penetrate the stratum corneum layer of the skin; and
(b) solid microspheres, an amount of the solid microspheres included in the skin care product having been selected to acoustically match the skin care product to the acoustic energy directed at the skin care product applied to the skin.

18. The cosmetic system of claim 14, wherein the applicator does not include any element extending beyond the distal face of the acoustic waveguide that would contact the user's skin while the applicator is in use.

19. The cosmetic system of claim 14, wherein the therapy head exhibits a triangular form factor, to facilitate positioning the distal face of the waveguide on skin surfaces having limited accessibility.

20. The cosmetic system of claim 14, wherein the distal face of the waveguide has a durometer and texture approximating that of human skin.
